(19) United States
(12) Patent Application Publication
(10) Pub. No.: US 2022/0260592 A1
(43) Pub. Date: Aug. 18, 2022

(54) MULTIPLEX ASSAY FOR DETERMINING THE ß-AMYLOID 42/40 RATIO IN HUMAN PLASMA SPECIMENS

(71) Applicant: QUEST DIAGNOSTICS INVESTMENTS LLC, Secaucus, NJ (US)

(72) Inventors: Keith R. MORNEAU, Greenville, NH (US); Brian G. SANSOUCY, Holden, MA (US)

(73) Assignee: QUEST DIAGNOSTICS INVESTMENTS LLC, Secaucus, NJ (US)

(21) Appl. No.: 17/610,044

(22) PCT Filed: May 8, 2020
(86) PCT No.: PCT/US2020/032010

§ 371 Date: Nov. 9, 2021

Related U.S. Application Data
(60) Provisional application No. 62/846,565, filed on May 10, 2019.

(51) Int. Cl. G01N 33/68; G01N 1/34; G01N 33/543

(52) U.S. Cl. CPC G01N 33/6896; G01N 1/34; G01N 2800/50; G01N 2800/52; G01N 33/543

(57) ABSTRACT

The present technology relates to methods for diagnosing, monitoring the progression of, assessing the efficacy of treatment of, or assessing risk for development of a neurodegenerative disorder in a patient. These methods are based on determining the ratio of β-amyloid 42 (“Aβ42”) to β-amyloid 40 (“Aβ40”) in a body fluid sample collected from a patient who has or is suspected of having a neurodegenerative disorder, using an improved and highly sensitive multiplex protein assay that simultaneously detects Aβ42 and Aβ40.

### CROSS-REFERENCE TO RELATED APPLICATIONS

This application claims priority under 35 U.S.C. § 119(e) to U.S. Provisional Application No. 62/846,565, filed May 10, 2019, which is hereby incorporated by reference in its entirety.

### TECHNICAL FIELD

The present technology relates to methods for diagnosing, monitoring the progression of, assessing efficacy of the treatment of, or assessing risk for development of a neurodegenerative disorder in a patient. These methods are based on determining the ratio of β-amyloid 42 (“Aβ42”) to β-amyloid 40 (“Aβ40”) in a body fluid sample collected from a patient who has or is suspected of having a neurodegenerative disorder, using an improved and highly sensitive multiplex protein assay method that simultaneously detects Aβ42 and Aβ40.

### BACKGROUND

The following description of the background of the present technology is provided simply as an aid in understanding the present technology and is not admitted to describe or constitute prior art to the present technology.

### Alzheimer's Disease

Neurodegenerative disorders are a major public health problem worldwide. For example, as of 2015, dementia was estimated to affect 46 million people, and this number is expected to rise to 131.5 million by 2050. Alzheimer's disease (AD) accounts for an estimated 60-70% of all dementia cases. (See, e.g., Fandos et al., 8 Alzheimer's & Dementia: Diagnosis, Assessment & Disease Monitoring 179 (2017).) Alzheimer's disease is characterized by dementia that typically begins with subtle and poorly recognized failure of memory that slowly becomes more severe and, eventually, incapacitating. Other common symptoms include confusion, poor judgment, language disturbance, agitation, withdrawal, and hallucinations. Occasionally, seizures, Parkinsonian features, increased muscle tone, myoclonus, incontinence, and mutism occur. Death usually results from general inanition, malnutrition, and pneumonia. The typical clinical duration of the disease is 8 to 10 years, with a range from 1 to 25 years. Approximately 25% of all AD is familial, of which approximately 95% is late-onset (age >60-65 years) and 5% is early-onset (age <65 years). See Thomas D. Bird, Alzheimer Disease Overview, in GENEREVIEWS® (M. P Adam, H. H. Ardinger & R. A. Pagon et al., eds.) (Oct. 23, 1998, last updated Dec. 20, 2018), ncbi.nlm.nih.gov/books/NBK1161/.

Because AD imposes an enormous economic and social burden, successful therapeutic interventions that can slow, stop, or prevent development of AD present a clear benefit. However, effective therapies for AD are currently unavailable, partly because individuals typically targeted in clinical trials evidence advanced neurodegenerative stages. AD diagnosis currently relies on clinical-neuropathologic assessment. Neuropathologic findings of β-amyloid plaques and intraneuronal neurofibrillary tangles remain the gold standard for diagnosis, despite the fact that it has shown sensitivities ranging from 70.9% to 87.3%, and specificities from 44.3% to 70.8%. (See Beach et al., 71 J. Neuropathol. Exp. Neurol. 266 (2012); Fandos et al., 8 Alzheimer's & Dementia: Diagnosis, Assessment & Disease Monitoring 179, 180 (2017).) Such clinical diagnosis of AD, based on signs of slowly progressive dementia and findings of gross cerebral cortical atrophy on neuroimaging, is correct in approximately 80-90% of cases. However, these screening methods are not useful for early AD detection and intervention because they focus on individuals who show symptoms of advanced neurodegeneration.

Effective AD treatments, on the other hand, will likely depend on early detection and intervention in asymptomatic (preclinical) or prodromal individuals. Thus, there is a need for methods that effectively detect the onset of AD in the absence of clinical-neuropathological symptoms.

### SUMMARY OF THE TECHNOLOGY

In one aspect, the present disclosure relates to a method for preparing a body fluid sample for detection of at least one of β-amyloid 42 (“Aβ42”) to β-amyloid 40 (“Aβ40”), comprising: obtaining a body fluid sample from a subject; and disassociating at least one of Aβ42 and Aβ40 within the body fluid sample from endogenous proteins by incubating the body fluid sample in a buffer solution comprising: a buffer; and a protein-compatible surfactant, wherein the body fluid sample is incubated in the buffer solution for at least 30 minutes.

In some embodiments, the buffer solution may comprise between 0.005 vol.-% and 5.0 vol.-%, or between 0.05 vol.-% and 0.5 vol.-%, of the protein-compatible surfactant. In some embodiments, the protein-compatible surfactant may comprise polysorbate 20, Triton X-100, or mixtures thereof. In some embodiments, the body fluid sample may be diluted in the buffer solution by a factor of between about 4 and about 16, by a factor of between about 8 and about 16, or by a factor of approximately 10. In some embodiments, the body fluid sample may be incubated in the buffer solution for at least approximately 30 minutes but no more than approximately 4 hours.

In some embodiments, the body fluid may be selected from the group consisting of blood, plasma, serum, lymphatic fluid, cerebrospinal fluid, synovial fluid, urine, and saliva. In some embodiments, the body fluid may be plasma.

In some embodiments, the method according to the present disclosure may further comprise performing an immunoassay on the body fluid sample after incubating the body fluid sample in the buffer solution to determine the concentration of at least one of Aβ42 and Aβ40.

In some embodiments, the method according to the present disclosure may further comprise determining the concentration of Aβ42 and Aβ40, and calculating the ratio of Aβ42 and Aβ40 in the body fluid sample. In some embodiments, calculating the ratio of Aβ42 and Aβ40 may comprise: calculating a dose (D) of Aβ42 from at least the first detectable signal; calculating a dose (D) of Aβ40 from at least the second detectable signal; and correcting the doses (D) of Aβ42 and Aβ40 to determine concentrations of Aβ42 and Aβ40 in the body fluid. In some embodiments, the concentration of Aβ42 in the body fluid may be determined from the dose (D) according to the relationship:

$$[ AB 42 ( \frac{pg}{ml} ) ] = {( {C}_{1} D )}^{{C}_{2}} ;$$

and the concentration of Aβ40 in the body fluid may be determined from the dose (D) according to the relationship:

$$[ AB 40 ( \frac{pg}{ml} ) ] = ( {C}_{3} ) ( D ) + D ,$$

wherein C₁, C₂, and C₃ are correction factors. In some embodiments, C₁ may be approximately 2.4271, C₂ may be approximately 0.9196, and C₃ may be approximately 0.35.

In some embodiments according to the present disclosure, the immunoassay may comprise an ELISA. In some embodiments, the immunoassay may be a digital ELISA.

In some embodiments according the present disclosure, the subject may have a neurodegenerative disorder, may be suspected of having a neurodegenerative disorder, may be undergoing treatment for a neurodegenerative disorder, may have a risk of developing a neurodegenerative disorder, or may be suspected of having a risk of developing a neurodegenerative disorder. In some embodiments, the neurodegenerative disorder may be selected from the group consisting of dementia, Alzheimer's Disease, and traumatic brain injury. In some embodiments, the neurodegenerative disorder may be Alzheimer's disease.

In another aspect, the present disclosure relates to a method for determining the ratio of Aβ42 to Aβ340 in a body fluid, comprising: preparing a body fluid sample for detection of at least one of Aβ42 and Aβ40 to produce free peptides by: obtaining a body fluid sample from a subject; and disassociating at least one of Aβ42 and Aβ40 within the body fluid sample from endogenous proteins by incubating the body fluid sample in a buffer solution comprising: a buffer; and a protein-compatible surfactant, wherein the body fluid sample is incubated in the buffer solution for at least 30 minutes; and performing an immunoassay on the body fluid sample, wherein concentrations of Aβ42 and Aβ40 in the body fluid sample are determined simultaneously from a single multiplex assay.

In some embodiments, the buffer solution may comprise between 0.005 vol.-% and 5.0 vol.-%, or between 0.05 vol.-% and 0.5 vol.-%, of the protein-compatible surfactant. In some embodiments, the protein-compatible surfactant may comprise polysorbate 20, Triton X-100, or mixtures thereof. In some embodiments, the body fluid sample may be diluted in the buffer solution by a factor of between about 4 and about 16, by a factor of between about 8 and about 16, or by a factor of approximately 10. In some embodiments, the body fluid sample may be incubated in the buffer solution for at least approximately 30 minutes but no more than approximately 4 hours.

In some embodiments, the body fluid may be selected from the group consisting of blood, plasma, serum, lymphatic fluid, cerebrospinal fluid, synovial fluid, urine, and saliva. In some embodiments, the body fluid may be plasma.

In some embodiments, the step of performing an immunoassay further comprises: measuring a first detectable signal from Aβ42 immunocomplexes; measuring a second detectable signal from Aβ40 immunocomplexes; calculating a dose (D) of Aβ42 from at least the first detectable signal; calculating a dose (D) of Aβ40 from at least the second detectable signal; and correcting the doses (D) of Aβ42 and Aβ40 to determine concentrations of Aβ42 and Aβ40 in the body fluid.

In some embodiments, the step of performing an immunoassay may further comprise: measuring a first detectable signal from Aβ42 immunocomplexes; measuring a second detectable signal from Aβ40 immunocomplexes; measuring a third detectable signal from product molecules, wherein the product molecules comprise reaction products from the reaction of substrate molecules with labeled Aβ42 or Aβ40 immunocomplexes, wherein the labeled immunocomplexes are derived from the body fluid sample; calculating a dose (D) of Aβ42 from at least the first detectable signal and the third detectable signal; calculating a dose (D) of Aβ40 from at least the second detectable signal and the third detectable signal; and correcting the doses (D) of Aβ42 and Aβ40 to determine concentrations of Aβ42 and Aβ40 in the body fluid.

In some embodiments according to the present disclosure, the step of performing an immunoassay may further comprise, before measuring a first detectable signal and after preparing a body fluid sample for detection of at least one of Aβ42 and Aβ40 to produce free peptide molecules: incubating free peptide molecules in solution with detector reagent molecules and capture agents, the capture agents comprising Aβ42 capture agents and Aβ40 capture agents, to produce Aβ42 immunocomplexes and Aβ40 immunocomplexes; washing the captured peptides to remove unbound or nonspecifically bound Aβ42 or Aβ40 and unbound or non-specifically bound detector reagent molecules; incubating the immunocomplexes with detectable label molecules, wherein the detectable label molecules bind to detector reagent molecules on the immunocomplexes, to produce labeled Aβ42 immunocomplexes and labeled Aβ40 immunocomplexes; washing the labeled immunocomplexes to remove unbound or non-specifically bound detectable label molecules; immobilizing the labeled immunocomplexes onto an assay disc in the presence of substrate molecules, wherein the substrate molecules react with the labeled Aβ42 immunocomplexes or labeled Aβ40 immunocomplexes to produce product molecules, and wherein the product molecules emit a third detectable signal.

In some embodiments, the concentration of Aβ42 in the body fluid may be determined from the dose (D) according to the relationship:

$$[ AB 42 ( \frac{pg}{ml} ) ] = {( {C}_{1} D )}^{{C}_{2}} ;$$

and the concentration of Aβ40 in the body fluid may be determined from the dose (D) according to the relationship:

$$[ AB 40 ( \frac{pg}{ml} ) ] = ( {C}_{3} ) ( D ) + D ,$$

wherein C₁, C₂, and C₃ are correction factors. In some embodiments, C₁ may be approximately 2.4271, C₂ may be approximately 0.9196, and C₃ may be approximately 0.35.

In some embodiments, the immunoassay may comprise an ELISA. In some embodiments, the immunoassay may comprise a digital ELISA.

In some embodiments, the first detectable signal and second detectable signal may be fluorescence signals. In some embodiments, the third detectable signal may be a fluorescence signal. In some embodiments, the first detectable signal, second detectable signal, and third detectable signal may be fluorescence signals.

In some embodiments according to the present disclosure, the Aβ42 capture agents or the Aβ40 capture agents may comprise paramagnetic beads. In some embodiments, the capture agents may comprise Aβ42-specific or β40-specific antibodies or antigen-binding fragments attached to the surfaces of the paramagnetic beads.

In some embodiments according to the present disclosure, the assay disc may comprise wells. In some embodiments, immobilizing labeled immunocomplexes onto an assay disc may comprise immobilizing the labeled immunocomplexes or bare capture agents within the wells. In some embodiments, each well may be configured to contain no more than one labeled immunocomplex or one bare capture agent therein.

In some embodiments, immobilizing labeled immunocomplexes onto an assay disc may further comprise enclosing the labeled immunocomplexes in the presence of the substrate molecules, within the wells, under an oil layer.

In another aspect, the present disclosure relates to a method of detecting, monitoring the progression of, assessing the efficacy of a treatment for, or assessing risk for development of a neurodegenerative disorder in a subject comprising any of the above-disclosed methods. In some embodiments, the neurodegenerative disorder may be selected from the group consisting of dementia, Alzheimer's Disease, and traumatic brain injury. In some embodiments, the neurodegenerative disorder may be Alzheimer's Disease.

In some embodiments, the subject may have a neurodegenerative disorder, may be suspected of having a neurodegenerative disorder, may be undergoing treatment for a neurodegenerative disorder, may have a risk of developing a neurodegenerative disorder, or may be suspected of having a risk of developing a neurodegenerative disorder.

In another aspect, the present disclosure relates to a method for determining the ratio of β-amyloid 42 (“Aβ42”) to (β-amyloid 40 (“Aβ40”) in a body fluid, comprising: (i) providing a body fluid sample; (ii) incubating the body fluid sample in a buffer solution comprising a protein-compatible surfactant for at least 30 minutes to produce free peptides; and (iii) performing an immunoassay on the body fluid sample. In some embodiments of the method, the concentrations of Aβ42 and Aβ40 in the body fluid sample may be determined simultaneously from a single multiplex immunoassay.

In some embodiments of the method, the ratio of Aβ42 to Aβ40 may be determined from a body fluid selected from the group consisting of blood, plasma, serum, lymphatic fluid, cerebrospinal fluid, synovial fluid, urine, and saliva. In a preferred embodiment, the body fluid may be plasma.

In some embodiments of the method, the body fluid sample may be incubated in a buffer solution comprising a protein-compatible buffer. In preferred embodiments, the protein-compatible surfactant may be selected from the group consisting of polysorbate 20, Triton X-100, or mixtures thereof. In particularly preferred embodiments, the buffer solution may comprise between 0.005 vol.-% and 5.0 vol.-% of a protein-compatible surfactant, more preferably between 0.05 vol.-% and 0.5 vol.-% of a protein-compatible surfactant.

In some embodiments of the method, incubating the body fluid sample in a buffer solution may further comprise diluting the body fluid sample in the buffer solution by a factor of between about 4 and about 16, preferably by a factor of between about 8 and about 16, and even more preferably by a factor of approximately 10. In some embodiments of the method, the body fluid sample may be incubated in the buffer solution for at least approximately 30 minutes but no more than approximately 4 hours.

In some embodiments of the method, the immunoassay may comprise an ELISA. In preferred embodiments, the immunoassay may comprise a digital ELISA. In more preferred embodiments of the method, the immunoassay may be performed using a Quanterix SIMOA® HD-1 analyzer.

In another aspect, the present technology provides a method for determining the ratio of Aβ42 to Aβ40 in a body fluid, comprising: (i) providing a body fluid sample; (ii) incubating the body fluid sample in a buffer solution comprising a protein-compatible surfactant for at least 30 minutes to produce free peptides; (iii) performing an immunoassay on the body fluid sample, wherein performing an immunoassay may further comprise: (iv) measuring a first detectable signal from Aβ42 immunocomplexes; (v) measuring a second detectable signal from Aβ40 immunocomplexes; (vi) calculating a dose (D) of Aβ42 from at least the first detectable signal; (vii) calculating a dose (D) of Aβ40 from at least the second detectable signal; and (viii) correcting the doses (D) of Aβ42 and Aβ40 to determine concentrations of Aβ42 and Aβ40 in the body fluid.

In another aspect, the present technology provides a method for determining the ratio of Aβ42 to Aβ40 in a body fluid, comprising: (i) providing a body fluid sample; (ii) incubating the body fluid sample in a buffer solution comprising a protein-compatible surfactant for at least 30 minutes to produce free peptides; (iii) performing an immunoassay on the body fluid sample, wherein performing an immunoassay may further comprise: (iv) measuring a first detectable signal from Aβ42 immunocomplexes; (v) measuring a second detectable signal from Aβ40 immunocomplexes; (vi) measuring a third detectable signal from product molecules, wherein the product molecules may comprise reaction products from the reaction of substrate molecules with labeled Aβ42 or Aβ40 immunocomplexes, wherein the labeled immunocomplexes may be derived from the body fluid sample; (vii) calculating a dose (D) of Aβ42 from at least the first detectable signal and the third detectable signal; (viii) calculating a dose (D) of Aβ40 from at least the second detectable signal and the third detectable signal; and (ix) correcting the doses (D) of Aβ42 and Aβ40 to determine concentrations of Aβ42 and Aβ40 in the body fluid.

In some embodiments of the method, the concentration of Aβ42 in the body fluid may be determined from the dose (D) according to the relationship:

$$[ AB 42 ( \frac{pg}{ml} ) ] = {( {C}_{1} D )}^{{C}_{2}} ;$$

and the concentration of Aβ40 in the body fluid may be determined from the dose (D) according to the relationship:

$$[ AB 40 ( \frac{pg}{ml} ) ] = ( {C}_{3} ) ( D ) + D ,$$

wherein C₁, C₂, and C₃ are correction factors. In preferred embodiments of the method, the correction factors may be as follows: C₁ may be approximately 2.4271; C₂ may be approximately 0.9196; and C₃ may be approximately 0.35.

In some embodiments of the method, the first detectable signal and second detectable signal may comprise fluorescence signals. In other embodiments, the first detectable signal, second detectable signal, and third detectable signal may comprise fluorescence signals.

In another aspect, the present technology provides a method for determining the ratio of Aβ42 to Aβ40 in a body fluid, comprising: (i) providing a body fluid sample; (ii) incubating the body fluid sample in a buffer solution comprising a protein-compatible surfactant for at least 30 minutes to produce free peptides; (iii) performing an immunoassay on the body fluid sample, wherein performing an immunoassay may further comprise: (iv) incubating free peptide molecules in solution with detector reagent molecules and capture agents, the capture agents comprising Aβ42 capture agents and Aβ40 capture agents, to produce Aβ42 immunocomplexes and Aβ40 immunocomplexes; (v) washing the captured peptides to remove unbound or nonspecifically bound Aβ42 or Aβ40 and unbound or non-specifically bound detector reagent molecules; (vi) incubating the immunocomplexes with detectable label molecules, wherein the detectable label molecules bind to detector reagent molecules on the immunocomplexes, to produce labeled Aβ42 immunocomplexes and labeled Aβ40 immunocomplexes; (vii) washing the labeled immunocomplexes to remove unbound or non-specifically bound detectable label molecules; (viii) immobilizing the labeled immunocomplexes onto an assay disc in the presence of substrate molecules, wherein the substrate molecules may react with the labeled Aβ42 immunocomplexes or labeled Aβ40 immunocomplexes to produce product molecules, wherein the product molecules emit a third detectable signal; (ix) measuring a first detectable signal from Aβ42 immunocomplexes; (x) measuring a second detectable signal from Aβ40 immunocomplexes; (xi) measuring a third detectable signal from product molecules, wherein the product molecules may comprise reaction products from the reaction of substrate molecules with labeled Aβ42 or Aβ40 immunocomplexes, wherein the labeled immunocomplexes may be derived from the body fluid sample; (xii) calculating a dose (D) of Aβ42 from at least the first detectable signal and the third detectable signal; (xiii) calculating a dose (D) of Aβ40 from at least the second detectable signal and the third detectable signal; and (xiv) correcting the doses (D) of Aβ42 and Aβ40 to determine concentrations of Aβ42 and Aβ40 in the body fluid.

In some embodiments of the method, the Aβ42 capture agents or the Aβ40 capture agents may comprise paramagnetic beads. In preferred embodiments of the method, the capture agents may comprise Aβ42-specific or Aβ40-specific antibodies or antigen-binding fragments attached to the surfaces of paramagnetic beads. In more preferred embodiments, the paramagnetic beads may be approximately 2.7 μm in diameter.

In some embodiments of the method, the assay disc may comprise wells, wherein immobilizing labeled immunocomplexes onto an assay disc may further comprises immobilizing the labeled immunocomplexes or bare capture agents within the wells. In preferred embodiments, each well may be sized to contain no more than one labeled immunocomplex or one bare capture agent therein. In more preferred embodiments, each well may have a diameter of approximately 4.50 μm and a depth of approximately 3.25 βm. In some preferred embodiments, immobilizing labeled immunocomplexes onto an assay disc may further comprise enclosing the labeled immunocomplexes in the presence of the substrate molecules, within the wells, under an oil layer.

In some embodiments of the method, the detector reagent may comprise a biotinylated detector antibody or biotinylated antigen-binding fragment. In some embodiments of the method, the detectable label molecule may be an enzyme, preferably streptavidin-β-galactosidase. In preferred embodiments, the substrate may be resorufin-β-d-galactopyranoside.

In another aspect, the present technology provides methods for detecting, monitoring the progression of, assessing the efficacy of a treatment for, or assessing risk for development of a neurodegenerative disorder in an individual, comprising any of the above-described methods. In preferred embodiments, the neurodegenerative disorder may be selected from the group consisting of dementia, Alzheimer's Disease, and traumatic brain injury.

### BRIEF DESCRIPTION OF THE DRAWINGS

FIG. 1 is a flow chart showing the process steps in one embodiment of the method.

FIG. 2 is a schematic illustration of one embodiment of an immunoassay according to the present technology.

FIG. 3 shows a box plot summarizing the immunoassay performance for plasma specimens obtained from patients exhibiting normal cognitive function, early MCI, late MCI, and Alzheimer's Disease.

FIG. 4 shows a box plot summarizing immunoassay performance for plasma specimens obtained from Alzheimer's Disease and late MCI patients against early MCI and normal patients.

### DETAILED DESCRIPTION

### Definitions

“Body fluid” and “bodily fluid,” used interchangeably herein, refer to a fluid sample from a human, animal, or cell culture. Body fluids include, but are not limited to amniotic fluid, blood, cerebrospinal fluid, peritoneal fluid, plasma, pleural fluid, saliva, semen, serum, sputum, tears, and urine. In preferred embodiments, the body fluid or bodily fluid is human plasma.

“Beta amyloid peptides,” “β-amyloids,” and “Aβ peptides,” used interchangeably herein, refer to β-amyloid 1-40 (“Aβ40”) and β-amyloid 1-42 (“Aβ42”) (40 and 42 amino acid peptides, respectively). Aβ42 and Aβ40 are proteolytic products from the amyloid precursor protein (APP) that has gained attention as a biomarker correlating with AD onset, mild cognitive impairment, vascular dementia, and other cognitive disorders. Beta-secretase cleavage of APP initially results in the production of an APP fragment that is further cleaved by gamma-secretase at residues 40-42 to generate two main forms of β-amyloid, Aβ40 and Aβ42. (See, e.g., R. Vassar et al., 29 J. Neurosci. 12787 (2009).)

Accumulation of amyloid in the form of extracellular plaques is a neuropathological hallmark of AD and is believed to play a central role in the neurodegenerative process. Aβ40 is the major amyloid component in AD plaques and is thought to be an initiating factor for their formation. In healthy and disease states, Aβ40 is the most abundant form of the amyloid peptides in both cerebrospinal fluid (CSF) and plasma (10-20× more abundant than Aβ42). Recent studies suggest that a decrease in the ratio of Aβ42 to Aβ40 may indicate AD progression. See, e.g., K. Yaffe et al., 305 JAMA 261 (2011).

Substantial clinical validation has now been developed around disease relevance of cerebrospinal fluid (CSF) levels of Aβ42. See, e.g., S. Janelidze et al., 74 JAMA Neurol. 1492 (2017). Compared to CSF-based screening methods, a blood-based Aβ42 screen would be a less-invasive, more cost-effective technique for identifying individuals at risk of developing AD, for monitoring the progression of a neurodegenerative disorder, or for monitoring treatment of a neurodegenerative disorder. Accordingly, there is significant interest in measuring blood levels of Aβ42, as well as Aβ40. However, concentrations of Aβ42 in blood (typically in the single pg/ml range) are over 100-fold lower than in cerebrospinal fluid, requiring very high analytical sensitivity for its reliable measurement.

“Aβ42/Aβ40 ratio” or “42/40 ratio,” as used herein, refers to the ratio of Aβ42 to Aβ40 in a fluid sample (e.g., a body fluid sample, such as plasma, CSF, etc.).

“Free peptide” as used herein, means a β-amyloid peptide molecule that is fully dissociated from endogenous plasma proteins, is not bound to a capture agent, and may freely diffuse in solution, alone or associated with surfactant molecules. In the present technology, the β-amyloid peptide may be Aβ42 or Aβ40. Similarly, the term “free peptide solution” means a solution comprising such Aβ42 or Aβ40 peptides.

“Protein-compatible surfactant,” as used herein, means a surfactant that does not cause an undesired response in, or otherwise make unavailable for assay, a protein of interest (e.g., beta-amyloid peptides). In some cases, a “protein-compatible surfactant” may facilitate dissociation of target biomolecules (e.g., Aβ42 or Aβ40 peptides) from endogenous plasma proteins to make them available for assaying (e.g., by immunoassay using digital ELISA). Protein-compatible surfactants known in the art include, but are not limited to, polysorbate 20 (i.e., Tween-20) and Triton X-100.

“Capture agent,” as used herein, refers to a solid support that may selectively or specifically bind free β-amyloid peptides. The solid support may be any solid surface that comes into contact with a solution comprising Aβ peptides (e.g., polymer beads, paramagnetic beads, microspheres or microbeads, nanoparticles, nanowires, planar surfaces, etc.). In preferred embodiments, the solid support may display immunoglobulin-related compositions (e.g., antibodies or antigen-binding fragments) on its surface(s). In preferred embodiments, the immunoglobulin-related compositions may be Aβ42-specific or Aβ40-specific antibodies or antigen-binding fragments. In some embodiments, each capture agent may have between one and one million, preferably between 100,000 and 500,000, immunoglobulin-related compositions (e.g., antibodies or antigen-binding fragments) attached to each solid support surface.

“Captured peptide,” as used herein, means an Aβ42 or Aβ40 peptide molecule that is bound to a solid support, such as a Aβ42 capture agent or Aβ40 capture agent, respectively. In preferred embodiments, the captured Aβ42 or captured Aβ40 peptide may be coupled to the capture agent by a specific binding interaction with an Aβ42-specific or Aβ40-specific immunoglobulin-related composition (e.g., antibody or antigen-binding fragment).

“Bare capture agent,” as used herein, means a capture agent which has not bound to a free peptide molecule. For example, a “bare Aβ42 capture agent” is an Aβ42 capture agent that has not captured an Aβ42 peptide, and a “bare Aβ40 capture agent” is an Aβ40 capture agent that has not captured an Aβ40 peptide. Because bare capture agents do not include a captured peptide, they may not form immunocomplexes or labeled immunocomplexes.

“Detector reagent,” as used herein, means a selective binding agent that may specifically or selectively bind to a captured Aβ42 or Aβ40 peptide. The detector reagent may be an immunoglobulin-related composition (e.g., antibody or antigen-binding fragment). These binding agents may selectively or specifically bind to captured Aβ42 or captured Aβ40. These binding agents may be naturally occurring or synthetic. In preferred embodiments, the detector reagent may be biotinylated antibodies or biotinylated antigen-binding fragments.

“Immunocomplex,” as used herein, means a capture agent bound to a captured peptide, which is in turn bound to a detector reagent molecule. In some embodiments, the “immunocomplex” may comprise a captured peptide, which may comprise a capture agent selectively or specifically bound to an Aβ42 or Aβ40 peptide molecule through an immunoglobulin-related composition (e.g., antibody or antigen-binding fragment) attached to the surface of the solid support of a capture agent. A captured peptide may in turn selectively bind to a detector reagent molecule. In preferred embodiments, the detector reagent may selectively or specifically bind to the captured peptide. More preferably, the detector reagent may be an immunoglobulin-related composition (e.g., antibody or antigen-binding fragment) that selectively binds to the captured peptide.

“Detectable label,” as used herein means a molecule that specifically or selectively binds to an immunocomplex. In preferred embodiments, a detectable label molecule may be any molecule that conjugates to the bound detector reagent moiety on an immunocomplex and either emits a detectable signal, complexes a substrate molecule which emits a detectable signal, or reacts with a substrate molecule to yield a product molecule that emits a detectable signal. In preferred embodiments, the detectable label molecule may comprise a linker moiety (e.g., avidin, streptavidin, or neutrAvidin moiety) that selectively binds to a complementary moiety of a bound detector reagent (e.g., biotin). In preferred embodiments, the detectable label molecule may be an enzyme. In preferred embodiments, the detectable label may be streptavidin-β-galactosidase (SBG).

“Labeled immunocomplex,” as used herein, means an immunocomplex with a detectable label molecule conjugated to the detector reagent moiety. For example, a “labeled Aβ42 immunocomplex” is an Aβ42 immunocomplex wherein the bound detector reagent may be conjugated to a detectable label molecule. Likewise, a “labeled Aβ40 immunocomplex” is an Aβ40 immunocomplex wherein the bound detector reagent may be conjugated to a detectable label molecule. In preferred embodiments, the bound detector reagent may be conjugated to a detectable label through a streptavidin-biotin linker.

“Trapped immunocomplex” or “immobilized immunocomplex,” as used herein, refers to a labeled Aβ42 immunocomplex or labeled Aβ40 immunocomplex that has been immobilized onto an assay disc (e.g., trapped within a well, in the presence of substrate molecules, under an oil layer). A “trapped immunocomplex” may react with substrate molecules trapped within the same well to produce products that emit a detectable signal (e.g., fluorescence).

“Substrate” or “substrate molecule,” as used herein, refers to a molecule upon which a detectable label molecule acts. As one example, a detectable label molecule may be an enzyme, which may participate in chemical reactions involving substrate molecules. A substrate molecule may bond with the enzyme active site, and an enzyme-substrate complex may be formed. The substrate may be transformed into one or more products, which may then be released from the active site, after which the active site is free to accept another substrate molecule. In the case of multiple substrates, the substrates may bind the active site in a particular order before reacting together to produce products. A substrate may be a radioisotope that may be complexed by a detectable label. A substrate is called “fluorogenic” if it gives rise to a fluorescent product when acted on by a detectable label molecule. A substrate is called “chromogenic” if it gives rise to a colored product when acted on by a detectable label molecule. A substrate molecule may also be a radioisotope, which may be complexed by a detectable label molecule.

“Specific binding” or “selective binding,” as used herein, refers to the activity of any agent, molecule, or compound that specifically or selectively binds to a peptide, detector reagent, or detectable label. For example, antibodies on Aβ42 capture agents or Aβ40 capture agents may specifically and selectively bind free Aβ42 or free Aβ40 peptide molecules, respectively, or specific portions thereof. Examples include, but are not limited to, antibodies or antibody fragments. These binding agents may be naturally occurring or synthetic.

“Antibody,” as used herein, refers to a protein consisting of one or more polypeptides substantially encoded by immunoglobulin genes or fragments of immunoglobulin genes. The recognized immunoglobulin genes may include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

“Enzyme linked immunosorbent assay” or “ELISA,” as used herein, refers to an antibody-based assay in which detection of the antigen of interest is accomplished via an enzymatic reaction producing a detectable signal. ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex.

In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase, horseradish peroxidase, or streptavidin-β-galactosidase (SBG), all of which generate a detectable product upon digestion of appropriate substrates.

“Single-molecule immunoassay,” “SIMOA®,” or “digital ELISA,” as used herein, refer to assay technology which allows for detecting thousands of single protein molecules simultaneously using the same reagents as conventional ELISA methods. “Digital ELISA” is a promising platform for detecting peptides in the pg/ml range. Described as “single molecule array” (SIMOA®) technology, this approach uses arrays of femtoliter-sized reaction chambers (wells) that can isolate and detect single protein molecules. The well volumes are approximately 2 billion times smaller than in conventional ELISA, permitting a rapid buildup of fluorescent product when an enzyme-labeled analyte protein is present. Due to the extremely small well volumes, which prevent fluorophore products from diffusing out of the wells, there exists a high local concentration of confined fluorescent substrate molecules within each reaction chamber. This high local fluorophore concentration is readily observed, such that only a single molecule is required to reach the detection limit. See, e.g., U.S. Pat. No. 8,846,415; Rissin et al., 28 Nat. Biotechnol. 595 (2010).

If a particular well contains a labeled immunocomplex (comprising a captured peptide), then the confined substrate molecules may be converted by the detectable label to products (e.g., “fluorophores”) and confined to a volume of approximately 40 femtoliters, generating high local product concentration and emitting a detectable signal (e.g., fluorescence). If a particular well contains a bare capture agent (with no captured peptide), then it will not contain a detectable label. Therefore, the well will not exhibit a detectable signal from product molecules. SIMOA® therefore allows protein concentration to be determined digitally and is termed “digital ELISA.” The ratio of the number of wells containing an immunocomplex to the total number of wells containing a bare capture agent corresponds to the sample analyte peptide concentration. See, e.g., U.S. Pat. No. 8,846,415; Rissin et al., 28 Nat. Biotechnol. 595 (2010).

“Detecting” or “detection,” as used herein, refers to determining the presence and/or concentration of a molecule in sample. In preferred embodiments, “detection” may be determining the presence of Aβ42 or Aβ40 in a bodily fluid sample. Detection does not require the method to provide 100% sensitivity.

“Detectable signal,” as used herein, means a quantifiable response to an environmental stimulus or a quantifiable emission of particles, light, or energy. A detectable signal may be optical (e.g., luminescence, chemiluminescence, fluorescence, or colorometric). A detectable signal may also be radioemission (e.g., from a radioisotope).

“Fluorophore,” as used herein refers to a molecule that absorbs light at a particular wavelength (excitation frequency) and subsequently emits light of a longer wavelength (emission frequency).

“Dose” or “uncorrected dose,” as used herein, refers to the calculated concentration of Aβ42 and/or Aβ40 within a body fluid sample. In a preferred embodiment, the dose may be determined using the Quanterix SIMOA® HD-1 Analyzer, using the SIMOA® software's 4PL (4-parameter logistic) regression via the following equation:

$$Y = E + \frac{A - E}{1 + {( \frac{D}{C} )}^{B}} , \qquad ( Equation 4 )$$

where:
A=the minimum value that can be obtained (i.e., detectable signal at 0 dose);
E=the maximum value that can be obtained (i.e., detectable signal at infinite dose);
C=the point of inflection (i.e., the point on the curve halfway between A and E);
B=Hill's slope of the curve (related to the steepness of the curve at point C);
Y=detectable signal from individual specimens
D=dose (pg/ml)

“Individual,” “patient,” or “subject,” as used herein, can be an individual organism, a vertebrate, a mammal, or a human. In preferred embodiments, the individual, patient, or subject is a human.

“Specificity,” as used herein in reference to the methods of the present technology, means the probability that a test result will be negative when a bare capture agent or no capture agent is immobilized within a particular well on an assay disc.

“Sensitivity,” as used herein in reference to the methods of the present technology, means the probability that a test result will be positive when a labeled Aβ42 immunocomplex or a labeled Aβ40 immunocomplex is immobilized within a particular well on an assay disc.

“About” or “approximately,” as used herein in reference to a number, is generally taken to include numbers that fall within a range of 1%, 5%, or 10% in either direction (greater than or less than) of the number unless otherwise stated or otherwise evident from the context (except where such number would be less than 0% or exceed 100% of a possible value).

### Multiplex Immunoassay for Detecting the 42/40 Ratio in Plasma

Conventional amyloid protein assay kits (e.g., Quanterix # 101995, Human Neurology 3-Plex Kit, Quanterix Corp., Lexington, Mass.) suffer from low recovery, making it difficult to accurately determine the 42/40 ratio. Modifying specimen dilution and incubation conditions, as well as applying analytical correction factors based on assay performance, enhances Aβ recovery and enables rapid, accurate determination of the 42/40 ratio from a single multiplex assay.

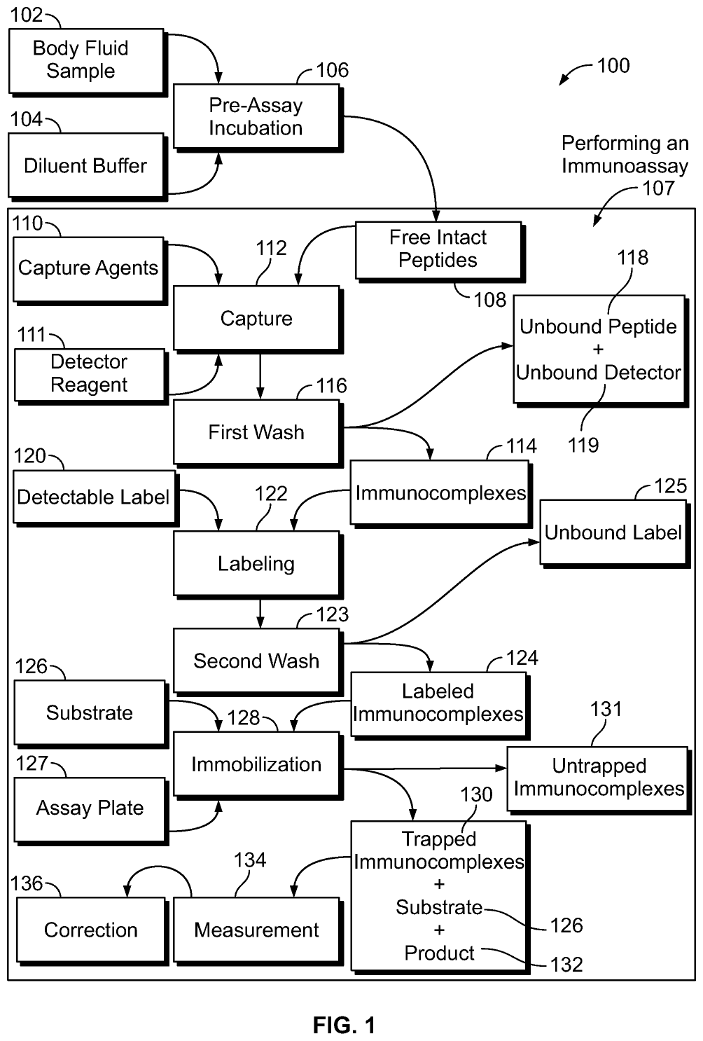

The present technology is best understood with reference to the figures. Referring to FIG. 1, some embodiments of the method 100 may comprise providing a body fluid sample 102. Body fluid sample 102 may comprise any body fluid (e.g., blood, plasma, serum, lymphatic fluid, cerebrospinal fluid, synovial fluid, urine, saliva, etc.) that contains, or is suspected of containing, Aβ42 and/or Aβ40 molecules. In preferred embodiments, body fluid sample 102 may comprise a human body fluid sample. In a preferred embodiment, body fluid sample 102 may comprise plasma.

Some embodiments of the method 100 may further comprise a pre-assay incubation 106, wherein body fluid sample 102 is diluted in a diluent buffer solution 104 to produce free peptides 108. Diluent buffer solution 104 may comprise any suitable protein-compatible buffer solution (e.g., phosphate-buffered saline). In a preferred embodiment, diluent buffer solution 104 may comprise Quanterix 4-Plex Diluent (Quanterix Corp., Lexington, Mass.).

Diluent buffer solution 104 may additionally comprise one or more surfactants capable of desorbing Aβ peptides (e.g., Aβ42 or A(340, etc.) from matrix plasma proteins to make AP peptides available for assaying. The surfactant(s) may comprise any suitable protein-compatible surfactant (e.g., polysorbate 20 (Tween-20), Triton X-100, or mixtures thereof, etc.). In a preferred embodiment, the surfactant comprises a mixture of Triton X-100 and Tween-20. In some embodiments, the surfactant may be present in a concentration of between 0.005 vol.-% and 5.0 vol.-%, preferably between 0.01 vol.-% and 1 vol.-%, and more preferably between 0.05 vol.-% and 0.5 vol.-%.

In some embodiments, pre-assay incubation 106 may comprise diluting body fluid sample 102 in diluent buffer solution 104 by a factor of between 1 and 20, preferably between 4 and 16, even more preferably between 8 and 12. In a preferred embodiment, body fluid sample 102 may be diluted in diluent buffer solution 104 by a factor of approximately 10.

In some embodiments, the pre-assay incubation 106 may take place over an extended time to allow disassociation of Aβ42 and Aβ40 from endogenous plasma proteins, thereby increasing Aβ42 and Aβ40 assay availability before performing an immunoassay on the body fluid sample. In preferred embodiments, pre-assay incubation 106 may comprise incubating the body fluid sample 102 in the diluent buffer solution 104 for between 1 min. and 480 min., preferably between 10 min. and 360 min., and more preferably between 30 min. and 240 min. In a preferred embodiment, the pre-assay incubation 106 takes place over at least 30 min. but for no more than 4 hours.

In some embodiments, the method 100 may further comprise, after the pre-assay incubation 106, performing an immunoassay 107, wherein the Aβ42 and Aβ40 peptides in the diluted body fluid sample are simultaneously assayed. Simultaneously assaying the Aβ42 and Aβ40 peptides may comprise any suitable assay method for determining peptide concentration (e.g., ELISA, digital ELISA, etc.). In a preferred embodiment, the assay method may comprise a digital ELISA.

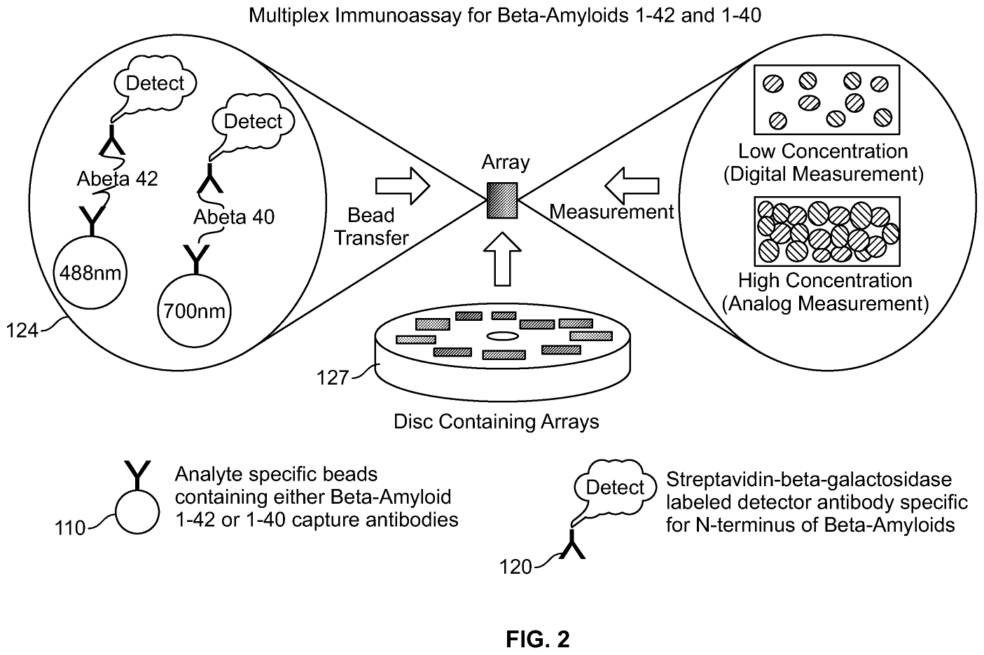

Referring now to FIGS. 1 and 2, in one embodiment, performing an immunoassay 107 may comprise capturing 112 free peptides 108 by incubating free peptides 108 in the presence of capture agents 110 and detector reagent molecule 111 to produce immunocomplexes 114. In preferred embodiments, the immunocomplexes may comprise “sandwich-type” complexes, in which a single capture agent may be bound to one or more AP peptide molecules at the C-terminus, and a detector reagent molecule may be bound to each capture Aβ peptide molecule at its N-terminus.

In some embodiments of the method 100, the capture agents 110 may comprise solid supports (e.g., beads, functionalized wells, microparticles, nanoparticles, etc.). In a preferred embodiment, the solid supports may comprise 2.7-μm-diameter paramagnetic beads.

In preferred embodiments, the capture agents may further comprise selective binding agents (e.g., Aβ42-specific binding agents or Aβ40-specific binding agents), which may be bound to the solid support. In a preferred embodiment the specific binding agents may comprise immunoglobulin-related compositions (e.g., Aβ42-specific antibodies or Aβ40-specific antibodies). In a particularly preferred embodiment, the support-bound Aβ42 antibodies or Aβ40 antibodies may bind selectively and specifically to either the Aβ42 or Aβ40 peptides, respectively, at the C-terminus.

In preferred embodiments, each capture agent may be functionalized with only one type of selective binding agent for AP peptides (e.g., for either Aβ42 or Aβ40). For example, each Aβ42 capture agent may be functionalized with Aβ42-specific antibodies (but not Aβ40-specific antibodies), while each Aβ40 capture agent may be functionalized with Aβ40-specific antibodies (but not Aβ42-specific antibodies).

Additionally, in preferred embodiments, each Aβ-specific capture agent may emit a distinct detectable signal (e.g., colorometric, luminescent, electroluminescent, radioemission, fluorescent, etc.). For example, each Aβ42 capture agent may emit a first fluorescence signal at a first wavelength, while each Aβ40 capture agent may emit a second fluorescence signal at a second wavelength.

In a preferred embodiment of the method, the capture agents 110 may comprise Quanterix SIMOA® Aβ42 Dye Encoded (488) Bead Concentrate (1.4×10⁹ beads/ml) (Quanterix # 102007, Quanterix Corp., Lexington, Mass.) and Quanterix SIMOA® Aβ40 Dye Encoded (700) Bead Concentrate (1.4×10⁹ beads/ml) (Quanterix # 102009, Quanterix Corp., Lexington, Mass.).

In some embodiments, the total number of capture agents in solution may outnumber free Aβ42 and Aβ40 molecules, together, by a factor of between 10,000 and 1, more preferably between 100 and 1. In a preferred embodiment, the total number of capture agents may outnumber the total number of free Aβ42 and free Aβ40 peptides, together, by approximately 10 to 1. Therefore, the captured peptide solution may comprise captured Aβ42, captured Aβ40, and bare capture agents.

Referring to FIGS. 1 and 2, in some embodiments, capturing 112 Aβ42 and Aβ40 peptides may further comprise incubating the free peptides 108 and capture agents 110 in the presence of detector reagent molecules 111. The detector reagent molecules may selectively or specifically bind to captured Aβ42 or captured Aβ40, to produce Aβ42 immunocomplexes or Aβ40 immunocomplexes, respectively. In a preferred embodiment, the detector reagent molecules may bind to either Aβ42 or Aβ40 through their common N-terminus sequence, such that the detector reagent molecules do not preferentially bind Aβ42 over Aβ40, or vice versa.

The detector reagent 111 molecules may be immunoglobulin-related compositions (e.g., antibodies or antigen-binding fragments). In a preferred embodiment, the detector reagent may comprise an immunoglobulin-related composition (e.g., antibody or antigen-binding fragment) that selectively binds to the common N-terminus of Aβ42 or Aβ40. In preferred embodiments of the method, the detector reagent may comprise a biotinylated antibody or biotinylated antigen-binding fragment. In a preferred embodiment of the method, the detector reagent may comprise Quanterix SIMOA® Aβ40/42 Biotinylated Detector Antibody (Quanterix #102010, Quanterix Corp., Lexington, Mass.).

In some embodiments, performing an immunoassay 107 may further comprise a first wash 116, wherein unbound or non-specifically bound peptides 118 and unbound or non-specifically bound detector reagent molecules 119 are removed from the assay solution. In a preferred embodiment, immunocomplexes 114 and bare capture agents (not shown) may be collected or retained for subsequent assay steps.

Referring still to FIGS. 1 and 2, performing an immunoassay 107 may further comprise labeling 122 the immunocomplexes 114 by incubating them in the presence of detectable label molecules 120. In some embodiments, one or more detectable label molecules conjugate to each immunocomplex through a linkage (e.g., streptavidin-biotin) to produce labeled immunocomplexes 124.

In preferred embodiments of the method, the detectable label 122 may comprise an enzyme (e.g., β-galactosidase, horseradish peroxidase (HRP), or alkaline phosphatase). In a preferred embodiment, the detectable label may comprise streptavidin-β-galactosidase (SBG). In a particularly preferred embodiment of the method, the detectable label may comprise SBG derived from a Quanterix Bulk SBG Kit (Quanterix # 101735, Quanterix Corp., Lexington, Mass.).

Referring still to FIG. 1, in some embodiments, performing an immunoassay 107 may further comprise a second wash 123, wherein unbound or non-specifically bound detectable label molecules 125 are removed from the assay solution. In a preferred embodiment, labeled immunocomplexes 124 and bare capture agents (not shown) may be collected or retained for subsequent assay steps.

In some embodiments of the method 100, performing an immunoassay 107 may further comprise immobilizing 128 labeled immunocomplexes 124 and bare capture agents (not shown) from solution onto an assay disc 127 in the presence of substrate molecules 126 to produce trapped immunocomplexes 130 for analysis. (Immobilization 128 may also trap bare capture agents (not shown).)

Referring to FIGS. 1 and 2, in some embodiments of the method 100, the assay disc 127 may comprise one or more arrays of wells. In preferred embodiments, wells in the test substrate may be sized to accommodate no more than one labeled immunocomplex or bare capture agent per well. In a particularly preferred embodiment of the method, the well dimensions may be approximately 4.25 μm wide by approximately 3.25 μm deep.

In some embodiments, labeled immunocomplexes 124 may be immobilized onto an assay disc 127 in the presence of substrate molecules 126. In preferred embodiments, the substrate may comprise any suitable substrate molecule 126 that may react with a detectable label molecule to produce product molecules 132 that emit a third detectable signal (e.g., fluorescence).

In a preferred embodiment, the substrate 126 may comprise resorufin-β-d-galactopyranoside (RGP). In a particularly preferred embodiment, the substrate molecules may be derived from an aliquot of Quanterix's Bulk RGP Kit (Quanterix # 101736, Quanterix Corp., Lexington, Mass.).

In some embodiments, immobilizing 128 labeled immunocomplexes 124 onto an assay disc 127 may comprise enclosing labeled immunocomplexes and bare capture agents within wells in an assay disc. In preferred embodiments, immobilizing labeled immunocomplexes onto an assay disc may further comprise spreading an oil (e.g., a Dupont Krytox® performance lubricant) across the assay disc, thereby enclosing immunocomplexes and bare capture agents in the wells in the presence of substrate molecules. In a preferred embodiment of the method, the sealing oil may be Quanterix SIMOA® Sealing Oil (Quanterix # 100206, Quanterix Corp., Lexington, Mass.).

In some embodiments, performing an immunoassay 107 may further comprise measuring detectable signals to determine the concentration of Aβ42 and Aβ40 in a body fluid sample. For example, in some embodiments, the Aβ42 capture agents may emit a first detectable signal (e.g., fluorescence), such that the number of labeled Aβ42 immunocomplexes and bare Aβ42 capture agents on the assay disc may be determined from measuring the first detectable signal. Additionally, in some embodiments, the Aβ40 capture agents may emit a second detectable signal (e.g., fluorescence), such that the number of labeled Aβ40 immunocomplexes and bare Aβ40 capture agents on the assay disc may be determined from measuring the second detectable signal. The first and second detectable signals may be measured by any suitable analyzer. In a preferred embodiment, the first and second detectable signals may be measured by digital fluorescence analyzer (e.g., a Quanterix SIMOA® HD-1 Analyzer).

Additionally, because labeled immunocomplexes 130 may be trapped on the assay disc 127 in the presence of substrate molecules 126, the substrate molecules may react with detectable label moieties on the labeled immunocomplexes 130 to produce product molecules 132 (e.g., fluorophores) that may emit a third detectable signal (e.g., fluorescence). Because the wells may be sealed, the product molecules may not be able to diffuse out of the wells, which may contain volumes on the order of femtoliters. Therefore, a high concentration of product molecules may build up within each well containing a labeled immunocomplex, making the third detectable signal (e.g., a fluorescence signal) readily observable. The third detectable signal may be measured by any suitable analyzer (e.g., a digital fluorescence analyzer, an analog fluorescence analyzer, a combination digital-analog fluorescence analyzer, etc.). In a preferred embodiment, the third detectable signal may be measured by digital fluorescence analyzer (e.g., a Quanterix SIMOA® HD-1 Analyzer).

In some embodiments, the first, second, and third detectable signals may be compared to determine the concentrations of Aβ42 and Aβ40 in a sample. The ratio of labeled immunocomplexes 130 to bare capture agents (not shown) on the assay disc 127 may indicate the concentration of Aβ42 and/or Aβ40 in the body fluid sample.

In a preferred embodiment, performing an immunoassay may further comprise measuring a detectable signal (e.g., radioemission, fluorescence, luminescence, or chemiluminescence, or colorometric signal) from product molecules in the assay disc. In preferred embodiments, the detectable signal may be a digital fluorescence signal. In a preferred embodiment, the fluorescence signal may be measured using a commercially-available analyzer. Most preferably, the commercially-available analyzer may be a Quanterix SIMOA® HD-1 analyzer (Quanterix Corp., Lexington, Mass.).

In one embodiment of the method, performing an immunoassay 107 may further comprise calculating a dose (D) (concentration of analyte), for Aβ42 and Aβ40 based on the relationship between the first, second, and third detectable signals. In a preferred embodiment, dose (D) may be calculated using a fitted calibration curve. In a preferred embodiment, dose (D) may be calculated using a 4-parameter logistic (“4PL”) fitted calibration curve via the following equation:

$$Y = E + \frac{A - E}{1 + {( \frac{D}{C} )}^{B}} , \qquad ( Equation 4 )$$

where:
A=the minimum value that can be obtained (i.e., detectable signal at 0 dose);
E=the maximum value that can be obtained (i.e., detectable signal at infinite dose);
C=the point of inflection (i.e., the point on the curve halfway between A and E);
B=Hill's slope of the curve (related to the steepness of the curve at point C);
Y=detectable signal from individual specimens
D=dose (pg/ml)

In a preferred embodiment, performing an immunoassay 127 may further comprise correcting 136 dose (D) values to determine concentrations of analyte peptides (e.g., Aβ42 and A(340). In a preferred embodiment, concentrations of Aβ42 and Aβ40 in the body fluid sample may be calculated from the dose (D), using correction factors. In a preferred embodiment, the concentrations of Aβ42 and Aβ40 (and the 42/40 ratio) in the body fluid sample may be calculated by correcting the dose using correction factors C₁, C₂, and C₃, according to the following equations:

$$\begin{matrix}[ A β42 ( \frac{pg}{ml} ) ] = {( {C}_{1} D )}^{{C}_{2}} & ( Equation 1 ) \\ [ A β40 ( \frac{pg}{ml} ) ] = ( ( {C}_{3} ) ( D ) + D ) & ( Equation 2 ) \\ 42 / 40 Ratio = \frac{[ A β42 ]}{[ A β40 ]} & ( Equation 3 )\end{matrix}$$

In a particularly preferred embodiment, C₁ may be approximately 2.4271, C₂ may be approximately 0.9196, and C₃ may be approximately 0.35.

The present disclosure also relates to methods for detecting, monitoring progression of, assessing efficacy of treatment for, or assessing risk for development of a neurodegenerative disorder in an individual. In some embodiments, the neurodegenerative disorder may be selected from the group consisting of dementia, Alzheimer's Disease, and traumatic brain injury. In a preferred embodiment of the method, the neurodegenerative disorder may be Alzheimer's Disease.

In one embodiment, the method may include determining the 42/40 ratio in a body fluid sample according to methods disclosed above, then comparing the 42/40 ratio to a reference value such that a 42/40 ratio greater than or equal to the reference value is within normal range and a 42/40 ratio less than the reference value is out of normal range. In one embodiment of the method, the reference value may be approximately 0.080, such that a 42/40 ratio greater than or equal to approximately 0.080 is within normal range, while a 42/40 ratio less than approximately 0.080 is out of normal range.

### EXAMPLES

### Example 1

Patient plasma samples are manually diluted in a diluent buffer solution to disassociate Aβ42 and Aβ40 from endogenous plasma proteins. Each patient sample is first thawed, then vortexed thoroughly. To achieve a 1:10 dilution, 30 μl of each patient sample is pipetted into a 1.5-ml snap-top tube containing 270 μl of Quanterix 4-Plex Diluent (Quanterix Corp., Lexington, Mass.). The diluted patient samples are allowed to equilibrate at room temperature for at least 30 minutes, but not more than 4 hours, before further processing.

Beta amyloid peptide controls are prepared from stock solutions of Aβ42 (e.g., β-Amyloid (Aβ) [1-42] (Human), Invitrogen # 03-112) and Aβ40 (e.g., Amyloid Beta Protein 1-40, Sigma Aldrich # A1075-1MG). From these stock solutions, “analog” and high-, medium-, and low-concentration controls are prepared for Aβ42 (100 pg/ml, 20 pg/ml, and 10 pg ml, respectively) and for Aβ40 (700 pg/ml, 150 pg/ml, and 70 pg/ml, respectively). Each control solution is diluted 1:10 (60 μl pipetted into 540 μl of diluent buffer solution (e.g., Quanterix 4-Plex Diluent)).

A series of calibrators is prepared from an Aβ42/Aβ40 (100/200 pg/ml) calibrator stock Solution, which is prepared by diluting Aβ42 calibrator concentrate (e.g., Aβ42 Calibrator Concentrate, Quanterix Corp., Lexington, Mass.) and Aβ40 calibrator concentrate (e.g., Aβ40 Calibrator Concentrate, Quanterix Corp., Lexington, Mass.) in diluent buffer solution (e.g., Quanterix 4-Plex Diluent) and stored at −15 ° C. to −25 ° C. To prepare the calibrators, diluent buffer solution (Quanterix 4-Plex Diluent) is pipetted into a series of 1.5-ml snap top tubes (333.3 μl into each tube). Calibrator stock is thawed, then thoroughly vortexed. Calibrator samples are then prepared by serial dilution of the calibrator stock in diluent buffer to achieve Aβ40/Aβ42 concentrations of 200/100 pg/ml, 66.7/33.3 pg/ml, 22.2/11.1 pg/ml, 7.41/3.70 pg/ml, 2.47/1.23 pg/ml, 0.82/0.41 pg/ml, 0.27/0.14 pg/ml, and 0/0 pg ml. 250 ul of each calibrator solution is pipetted into pre-determined positions in an assay disc.

Diluted control and patient samples are pipetted (250 μl of each) into pre-determined positions in an assay disc. Each calibrator, control, and patient sample (i.e., body fluid sample) is prepared and run in duplicate. The plates are then sealed with X-Pierce Sealing Films.

The immunoassay is then run using a Quanterix SIMOA® HD-1 Analyzer using a standard two-step “homebrew” protocol. Initial concentrations are calculated using a four-parameter logistic calibration curve, according to the following equation:

$$Y = E + \frac{A - E}{1 + {( \frac{D}{C} )}^{B}} , \qquad ( Equation 4 )$$

where:
A=the minimum value that can be obtained (i.e., detectable signal at 0 dose);
E=the maximum value that can be obtained (i.e., detectable signal at infinite dose);
C=the point of inflection (i.e., the point on the curve halfway between A and E);
B=Hill's slope of the curve (related to the steepness of the curve at point C); and
Y=detectable signal from individual specimens.
D=dose (pg/ml)

The raw concentration values are then corrected and used to calculate the 42/40 ratio according to the following equations:

$$\begin{matrix}[ A β42 ( \frac{pg}{ml} ) ] = {( {C}_{1} D )}^{{C}_{2}} & ( Equation 1 ) \\ [ A β40 ( \frac{pg}{ml} ) ] = ( ( {C}_{3} ) ( D ) + D ) & ( Equation 2 ) \\ 42 / 40 Ratio = \frac{[ A β42 ]}{[ A β40 ]} , & ( Equation 3 )\end{matrix}$$

where C₁=2.4271, C₂=0.9196, and C₃=0.35.

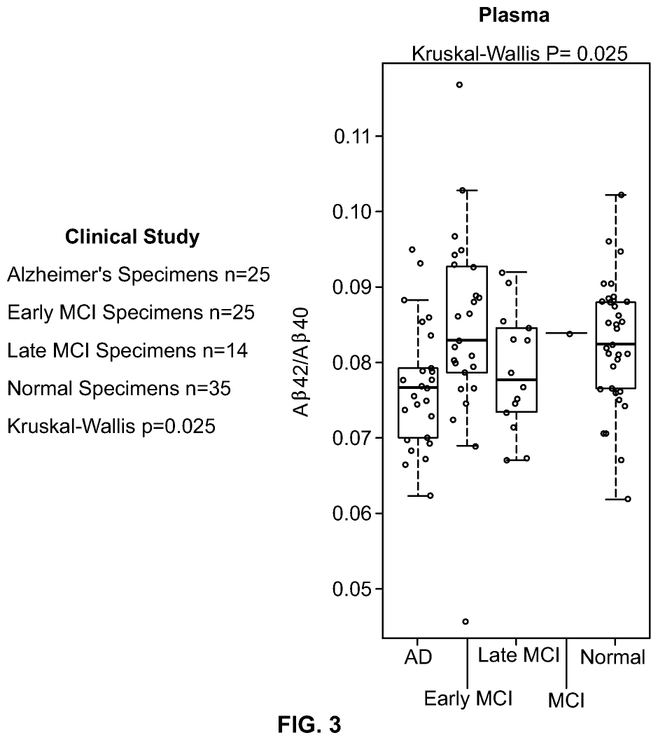

FIG. 3 shows a box plot summarizing the immunoassay performance for plasma specimens obtained from patients exhibiting normal cognitive function, early MCI, late MCI, and Alzheimer's Disease. The mean plasma 42/40 ratio measured for AD and late MCI patients is observably lower than that measured for AD and late MCI patients.

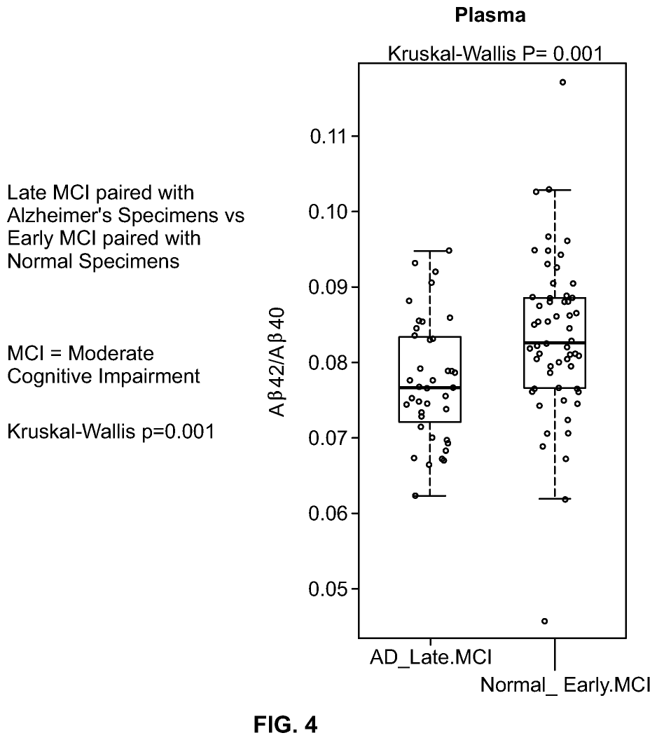

FIG. 4 shows a second box plot comparing immunoassay performance for AD patients, paired with late MCI patients, against early MCI patients, paired with normal patients. The mean plasma 42/40 ratio observed for the AD/late MCI patients is higher than that observed for normal/early MCI patients.

The multiplex method described herein assays Aβ42 and Aβ40 simultaneously with optimal recoveries that enable calculation of the 42/40 ratio from plasma, with proven clinical sensitivity of 76% and clinical specificity of 71%, as well as positive predictive value of 66% and negative predictive value of 81%. When employing correction factors to each analyte's baseline recovery, the probability statistic (obtained from a one-tailed, T-test) improves from p=0.011 without additional correction, to p=0.004 with application of the correction factors. This represents an improvement of approximately 36% in terms of analytical specificity and sensitivity.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method for preparing a body fluid sample for detection of at least one of β-amyloid 42 (“Aβ42”) to β-amyloid 40 (“Aβ40”), comprising:
obtaining a body fluid sample from a subject; and
disassociating at least one of Aβ42 and Aβ40 within the body fluid sample from endogenous proteins by incubating the body fluid sample in a buffer solution comprising:
a buffer; and
a protein-compatible surfactant,
wherein the body fluid sample is incubated in the buffer solution for at least 30 minutes.

2. The method of claim 1, wherein the buffer solution comprises between 0.005 vol.-% and 5.0 vol.-% of the protein-compatible surfactant.

3. The method of claim 1 or claim 2, wherein the body fluid sample is diluted in the buffer solution by a factor of between about 4 and about 16.

4. The method of any one of claims 1-3, wherein the protein-compatible surfactant comprises polysorbate 20, Triton X-100, or mixtures thereof

5. The method of any one of claims 1-4, wherein the body fluid is selected from the group consisting of blood, plasma, serum, lymphatic fluid, cerebrospinal fluid, synovial fluid, urine, and saliva.

6. The method of any one of claims 1-5, wherein the body fluid sample is incubated in the buffer solution for at least approximately 30 minutes but no more than approximately 4 hours.

7. The method of any one of claims 1-6 further comprising performing an immunoassay on the body fluid sample after incubating the body fluid sample in the buffer solution to determine the concentration of at least one of Aβ42 and Aβ40.

8. The method of claim 7 further comprising determining the concentration of Aβ42 and Aβ40, and calculating the ratio of Aβ42 and Aβ40 in the body fluid sample.

9. The method of claim 8, wherein calculating the ratio of Aβ42 and Aβ40 comprises:
calculating a dose (D) of Aβ42 from at least the first detectable signal;
calculating a dose (D) of Aβ40 from at least the second detectable signal; and
correcting the doses (D) of Aβ42 and Aβ40 to determine concentrations of Aβ42 and Aβ40 in the body fluid.

10. The method of claim 8 or claim 9, wherein the concentration of Aβ42 in the body fluid is determined from the dose (D) according to the relationship:
$$[ AB 42 ( \frac{pg}{ml} ) ] = {( {C}_{1} D )}^{{C}_{2}} ;$$
and
wherein the concentration of Aβ40 in the body fluid is determined from the dose (D) according to the relationship:
$$[ AB 40 ( \frac{pg}{ml} ) ] = ( {C}_{3} ) ( D ) + D ,$$
wherein C₁, C₂, and C₃ are correction factors.

11. The method of claim 10, wherein C₁ is approximately 2.4271, C₂ is approximately 0.9196, and C₃ is approximately 0.35.

12. The method of any one of claims 7-11, wherein the immunoassay comprises an ELISA.

13. The method of any one of claims 1-12, wherein the subject has a neurodegenerative disorder, is suspected of having a neurodegenerative disorder, is undergoing treatment for a neurodegenerative disorder, has a risk of developing a neurodegenerative disorder, or is suspected of having a risk of developing a neurodegenerative disorder.

14. A method for determining the ratio of Aβ42 to Aβ40 in a body fluid, comprising:
preparing a body fluid sample for detection of at least one of Aβ42 and Aβ40 according to the method of any one of claims 1-6 to produce free peptide molecules; and
performing an immunoassay on the body fluid sample,
wherein concentrations of Aβ42 and Aβ40 in the body fluid sample are determined simultaneously from a single multiplex assay.

15. The method of claim 14, wherein the step of performing an immunoassay further comprises:
measuring a first detectable signal from Aβ42 immunocomplexes;
measuring a second detectable signal from Aβ40 immunocomplexes;
calculating a dose (D) of Aβ42 from at least the first detectable signal;
calculating a dose (D) of Aβ40 from at least the second detectable signal; and
correcting the doses (D) of Aβ42 and Aβ40 to determine concentrations of Aβ42 and Aβ40 in the body fluid.

16. The method of claim 14, wherein the step of performing an immunoassay further comprises:
measuring a first detectable signal from Aβ42 immunocomplexes;
measuring a second detectable signal from Aβ40 immunocomplexes;
measuring a third detectable signal from product molecules, wherein the product molecules comprise reaction products from the reaction of substrate molecules with labeled Aβ42 or Aβ40 immunocomplexes, wherein the labeled immunocomplexes are derived from the body fluid sample;
calculating a dose (D) of Aβ42 from at least the first detectable signal and the third detectable signal;
calculating a dose (D) of Aβ40 from at least the second detectable signal and the third detectable signal; and
correcting the doses (D) of Aβ42 and Aβ40 to determine concentrations of Aβ42 and Aβ40 in the body fluid.

17. The method of any one of claims 14-16, wherein performing an immunoassay further comprises, before measuring a first detectable signal and after preparing a body fluid sample for detection of at least one of Aβ42 and Aβ40 to produce free peptide molecules:
incubating free peptide molecules in solution with detector reagent molecules and capture agents, the capture agents comprising Aβ42 capture agents and Aβ40 capture agents, to produce Aβ42 immunocomplexes and Aβ40 immunocomplexes;
washing the captured peptides to remove unbound or nonspecifically bound Aβ42 or Aβ40 and unbound or non-specifically bound detector reagent molecules;
incubating the immunocomplexes with detectable label molecules, wherein the detectable label molecules bind to detector reagent molecules on the immunocomplexes, to produce labeled Aβ42 immunocomplexes and labeled Aβ40 immunocomplexes;
washing the labeled immunocomplexes to remove unbound or non-specifically bound detectable label molecules;
immobilizing the labeled immunocomplexes onto an assay disc in the presence of substrate molecules, wherein the substrate molecules react with the labeled Aβ42 immunocomplexes or labeled Aβ40 immunocomplexes to produce product molecules, and wherein the product molecules emit a third detectable signal.

18. The method of any one of claims 14-17, wherein the concentration of Aβ42 in the body fluid is determined from the dose (D) according to the relationship:
$$[ AB 42 ( \frac{pg}{ml} ) ] = {( {C}_{1} D )}^{{C}_{2}} ;$$
and
wherein the concentration of Aβ40 in the body fluid is determined from the dose (D) according to the relationship:
$$[ AB 40 ( \frac{pg}{ml} ) ] = ( {C}_{3} ) ( D ) + D ,$$
wherein C₁, C₂, and C₃ are correction factors.

19. The method of claim 18, wherein C₁ is approximately 2.4271, C₂ is approximately 0.9196, and C₃ is approximately 0.35.

20. The method of any one of claims 14-19, wherein the immunoassay comprises an ELISA.

21. The method of any one of claims 14-19, wherein the first detectable signal and second detectable signal are fluorescence signals.

22. The method of any one of claims 14-20, wherein the third detectable signal is a fluorescence signal.

23. The method of any one of claims 14-21, wherein the Aβ42 capture agents or the Aβ40 capture agents comprise paramagnetic beads.

24. The method of any one of claims 14-22, wherein the capture agents comprise Aβ42-specific or Aβ40-specific antibodies or antigen-binding fragments attached to the surfaces of the paramagnetic beads.

25. The method of any one of claims 17-24, wherein:
the assay disc comprises wells;
immobilizing labeled immunocomplexes onto an assay disc comprises immobilizing the labeled immunocomplexes or bare capture agents within the wells; and
each well is configured to contain no more than one labeled immunocomplex or one bare capture agent therein.

26. The method of any one of claims 17-25, wherein immobilizing labeled immunocomplexes onto an assay disc further comprises enclosing the labeled immunocomplexes in the presence of the substrate molecules, within the wells, under an oil layer.

27. A method of detecting, monitoring the progression of, assessing the efficacy of a treatment for, or assessing risk for development of a neurodegenerative disorder in a subject, comprising the method of any one of claims 14-26.

28. The method of claim 27, wherein the neurodegenerative disorder is selected from the group consisting of dementia, Alzheimer's Disease, and traumatic brain injury.

29. The method of any one of claim 27 or claim 28, wherein the subject has a neurodegenerative disorder, is suspected of having a neurodegenerative disorder, is undergoing treatment for a neurodegenerative disorder, has a risk of developing a neurodegenerative disorder, or is suspected of having a risk of developing a neurodegenerative disorder.

30. A method for determining the ratio of Aβ42 to Aβ40 in a body fluid, comprising:
obtaining a body fluid sample from a subject;
incubating the body fluid sample in a buffer solution comprising a protein-compatible surfactant for at least 30 minutes to produce free peptides; and
performing an immunoassay on the body fluid sample,
wherein concentrations of Aβ42 and Aβ40 in the body fluid sample are determined simultaneously from a single multiplex assay.

31. The method of claim 30, wherein the step of performing an immunoassay further comprises:
measuring a first detectable signal from Aβ42 immunocomplexes;
measuring a second detectable signal from Aβ40 immunocomplexes;
calculating a dose (D) of Aβ42 from at least the first detectable signal;
calculating a dose (D) of Aβ40 from at least the second detectable signal; and
correcting the doses (D) of Aβ42 and Aβ40 to determine concentrations of Aβ42 and Aβ40 in the body fluid.

32. The method of claim 30, wherein the step of performing an immunoassay further comprises:
measuring a first detectable signal from Aβ42 immunocomplexes;
measuring a second detectable signal from Aβ40 immunocomplexes;
measuring a third detectable signal from product molecules, wherein the product molecules comprise reaction products from the reaction of substrate molecules with labeled Aβ42 or Aβ40 immunocomplexes, wherein the labeled immunocomplexes are derived from the body fluid sample;
calculating a dose (D) of Aβ42 from at least the first detectable signal and the third detectable signal;
calculating a dose (D) of Aβ40 from at least the second detectable signal and the third detectable signal; and
correcting the doses (D) of Aβ42 and Aβ40 to determine concentrations of Aβ42 and Aβ40 in the body fluid.

33. The method of claim 31 or claim 32, wherein performing an immunoassay further comprises, before measuring a first detectable signal and after incubating the body fluid sample in a buffer solution:
incubating free peptide molecules in solution with detector reagent molecules and capture agents, the capture agents comprising Aβ42 capture agents and Aβ40 capture agents, to produce Aβ42 immunocomplexes and Aβ40 immunocomplexes;
washing the captured peptides to remove unbound or nonspecifically bound Aβ42 or Aβ40 and unbound or non-specifically bound detector reagent molecules;
incubating the immunocomplexes with detectable label molecules, wherein the detectable label molecules bind to detector reagent molecules on the immunocomplexes, to produce labeled Aβ42 immunocomplexes and labeled Aβ40 immunocomplexes;
washing the labeled immunocomplexes to remove unbound or non-specifically bound detectable label molecules;
immobilizing the labeled immunocomplexes onto an assay disc in the presence of substrate molecules,
wherein the substrate molecules react with the labeled Aβ42 immunocomplexes or labeled Aβ40 immunocomplexes to produce product molecules, and
wherein the product molecules emit a third detectable signal.

34. The method of any one of claims 31-33,
wherein the concentration of Aβ42 in the body fluid is determined from the dose (D) according to the relationship:
$$[ AB 42 ( \frac{pg}{ml} ) ] = {( {C}_{1} D )}^{{C}_{2}} ;$$
and
wherein the concentration of Aβ40 in the body fluid is determined from the dose (D) according to the relationship:
$$[ AB 40 ( \frac{pg}{ml} ) ] = ( {C}_{3} ) ( D ) + D ,$$
wherein C₁, C₂, and C₃ are correction factors.

35. The method of claim 34, wherein C₁ is approximately 2.4271, C₂ is approximately 0.9196, and C₃ is approximately 0.35.

36. The method of any one of claims 30-35, wherein the body fluid is selected from the group consisting of blood, plasma, serum, lymphatic fluid, cerebrospinal fluid, synovial fluid, urine, and saliva, and is preferably plasma.

37. The method of any one of claims 30-36, wherein the protein-compatible surfactant comprises polysorbate 20, Triton X-100, or mixtures thereof

38. The method of any one of claims 30-37, wherein the buffer solution comprises between 0.005 vol.-% and 5.0 vol.-%, preferably between 0.05 vol.-% and 0.5 vol.-%, of the protein-compatible surfactant.

39. The method of any one of claims 30-38, wherein the body fluid sample is diluted by a factor of between about 4 and about 16, preferably by a factor of between about 8 and about 16, more preferably by a factor of approximately 10, in the buffer solution.

40. The method of any one of claims 30-39, wherein the body fluid sample is incubated in the buffer solution for at least approximately 30 minutes but no more than approximately 4 hours.

41. The method of any one of claims 30-40, wherein the immunoassay comprises an ELISA, preferably a digital ELISA.

42. The method of any one of claims 31-41, wherein the first detectable signal and second detectable signal are fluorescence signals.

43. The method of any one of claims 32-42, wherein the third detectable signal is a fluorescence signal.

44. The method of any one of claims 33-43, wherein the Aβ42 capture agents or the Aβ40 capture agents comprise paramagnetic beads.

45. The method of any one of claims 33-44, wherein the capture agents comprise Aβ42-specific or Aβ40-specific antibodies or antigen-binding fragments attached to the surfaces of the paramagnetic beads.

46. The method of any one of claims 33-45, wherein:
the assay disc comprises wells;
immobilizing labeled immunocomplexes onto an assay disc comprises immobilizing the labeled immunocomplexes or bare capture agents within the wells; and
each well is configured to contain no more than one labeled immunocomplex or one bare capture agent therein.

47. The method of any one of claims 30-46, wherein immobilizing labeled immunocomplexes onto an assay disc further comprises enclosing the labeled immunocomplexes in the presence of the substrate molecules, within the wells, under an oil layer.

48. A method of detecting, monitoring the progression of, assessing the efficacy of a treatment for, or assessing risk for development of a neurodegenerative disorder in a subject, comprising the method of any one of claims 30-47.

49. The method of claim 48, wherein the neurodegenerative disorder is selected from the group consisting of dementia, Alzheimer's Disease, and traumatic brain injury.

50. The method of claim 48 or claim 49, wherein the subject has a neurodegenerative disorder, is suspected of having a neurodegenerative disorder, or is suspected of having a risk of developing a neurodegenerative disorder.
